(19) United States
(12) Patent Application Publication
(10) Pub. No.: US 2013/0011297 A1
(43) Pub. Date: Jan. 10, 2013

(54) METHOD FOR STERILIZATION

(75) Inventors: Kieron D. Walsh, Westborough, MA (US); Asa Lagerlof, Uppsala (SE); Klaus Gebauer, Uppsala (SE)

(73) Assignee: GE HEALTHCARE BIO-SCIENCES CORP., PISCATAWAY, NJ (US)

(21) Appl. No.: 13/634,689

(22) PCT Filed: Mar. 14, 2011
(86) PCT No.: PCT/US11/28270

§ 371 Date: Sep. 13, 2012

Related U.S. Application Data
(60) Provisional application No. 61/317,888, filed on Mar. 26, 2010.

(51) Int. Cl. A61L 2/07

(52) U.S. Cl. 422/26

(57) ABSTRACT

The present invention relates to a method for providing a sterile connected device, especially of sensitive material, such as material not compatible with sterilization by gamma irradiation. The method comprises providing connectors and a tubing in an autoclave bag, and autoclaving the sealed autoclave bag containing the connectors and tubing for sterilization. Optionally, the connectors and tubing are assembled prior to autoclave. Also provided are sterile, connected devices sterilized according to the present method.

### CROSS-REFERENCE TO RELATED APPLICATIONS

This application claims priority to U.S. provisional patent application No. 61/317,888 filed Mar. 26, 2010; the disclosure of which is incorporated herein by reference in its entirety.

### FIELD OF THE INVENTION

The present invention relates to a method for providing a sterile connected device, especially made of sensitive materials. In particular, the invention relates to a method for providing a sterile connected device, by autoclave, which includes a component that is not compatible with sterilization by gamma irradiation.

### BACKGROUND OF THE INVENTION

The manufacture of biopharmaceuticals, particularly drugs based on bioactive molecules such as proteins, peptides and nucleic acids, requires the production and purification of these molecules on an industrial scale. For obvious reasons, it is of critical importance to ensure that the processes are conducted under sterile conditions and potentially harmful contaminants are removed from the system before use.

There are a number of products which have features that effectively eliminate the need to clean, sterilize, or validate multiple-use systems in the manufacturing process. For example, the READYTOPROCESS™ products (GE Healthcare, Westborough, Mass.) are designed to enable lean and responsive biopharmaceutical development and production with assured safety and cost-efficiency. Typical applications of READYTOPROCESS™ products include: aseptic clarification and purification of vaccines, monoclonal antibodies, recombinant proteins, and plasmids, aseptic cell processing, environments where terminal sterilization is not feasible, preclinical through Phase II clinical trials and fast-track drug development processes.

One of the challenges in providing sterile systems lies in providing sterile units of tubings and connectors. While many connectors can be gamma irradiated, available long-lasting, high pressure tubings generally can not be gamma irradiated due to the material used for such tubings. Furthermore, silicon based tubings can not be sealed using tubing welders, which itself does not embody the off the shelf assembly concept. Currently, these tubings are first autoclaved, then assembled with the gamma irradiated connecters following sterile procedure in for example a laminar flow hood. The method is cumbersome and there is a risk for contamination.

There is a need for methods which provided sterile units of tubings and connectors that are easy to perform and also minimizes the likelihood for contamination.

### SUMMARY OF THE INVENTION

The present invention provides a new sterilization method for providing a sterile connected device, especially made of sensitive materials. The inventors have found that a sterile connected device can be made by autoclave, when the device assembly includes a component that is not compatible with sterilization by gamma irradiation.

Thus, in a first aspect, there is provided a method for sterilization of connected device including a tubing and at least one connector. The method comprises first providing the connector and tubing in an autoclave bag, where the tubing is not compatible with sterilization by gamma irradiation; and then autoclaving the sealed autoclave bag which allows steam penetration to reach the interior surfaces of the connector and tubing. Optionally, the tubing is pump tubing suitable for high pressure or long term use which has a PTFE internal reinforcing layer. The connectors are gendered or genderless connectors.

In one embodiment, the tubing and connector(s) are not connected before autoclaving. In this embodiment, the method further comprises connecting the tubing with the connector(s) before opening the bag. Thus, a sealed unit is created such that the interior surfaces remain sterile upon opening of the autoclave bag.

In another embodiment, two connectors are in the autoclave bag, one of the connectors is connected to the tubing before autoclave. In this embodiment, the method further comprises connecting the tubing with the connectors before opening the bag. Thus, a sealed unit is created such that the interior surfaces remain sterile upon opening of the autoclave bag.

In yet another embodiment, two connectors are included in the autoclave bag, both connectors and the tubing are connected before autoclaving. In one variation, at least one of the connectors is breathable. In another variation, both connectors are non-breathable. In still another variation, the connectors are non-breathable, yet at least one includes a vent valve. The vent valve is open before autoclave, and is closed after autoclave, before the sterile connected device is taken out of the autoclave bag.

In another aspect, there is provided sterile, connected devices sterilized according to the present methods.

### DETAILED DESCRIPTION OF THE INVENTION

The present invention provides a new and efficient method for providing sterile connected devices including tubings and connectors. The method is easy to perform and also minimizes the likelihood for contamination. The method provides sterile ready to use parts that include components which are not gamma irradiation compatible. The devices are particularly useful in the manufacturing of biopharmaceuticals.

Thus, in a first aspect, the invention relates to a method for providing a sterile connected device including a tubing and at least one connector. First, the connectors and tubing are provided in an autoclave bag. Then the sealed autoclave bag is autoclaved. The autoclave bag allows steam penetration thus the autoclave step sterilizes the connectors and tubing. In one preferred embodiment, the tubing and the connectors are connected together before opening the autoclave bag. In another preferred embodiment, the tubing and the connectors are pre-connected before being put into the autoclave bag.

The invention provides sterile parts in a desired connection assembly (connected device) which are not compatible with gamma irradiation for pre-use sterilization or bioburden reduction. For example, high pressure pump tubings are essential components to allow high pressure applications (UF/DF) or applications that require long tubing life, such as continuous perfusion for cell culture in bag format, such as WAVE CELLBAG™, Yet, these pump tubings are not gamma irradiation compatible. One example of such pump tubing is GORE™ STA-PURE® tubing. These pump tubings are made of platinum-cured silicone which has a PTFE internal reinforcing layer, and is available in sizes up to 50 mm ID.

Furthermore, other components, such as sensors, could also be autoclaved and connected to desired connectors according to the method herein described. The method is particularly useful for component parts, when at least one of them is not compatible with gamma irradiation, yet may be easily assembled while still protected by the bag from contamination.

Preferably, the tubing has a restricted length, such as less than one meter in length. More preferably the tubing has a length less than half a meter long. As discussed in detail later, this autoclaved tubing is used in an assembly together with gamma irradiated parts, such as one or more connectors.

Suitable connectors include both gendered or genderless aseptic connectors. These connectors could be compatible with gamma irradiation. Alternatively, they could be non-compatible with gamma irradiation, as long as they are autoclavable. Optionally, the connectors are sterilized by gamma irradiation before autoclaving. It is preferable that after connection, the connected device (connection assembly) forms a sealed unit such that the unit remains sterile upon opening of the autoclave bag. Examples of suitable connectors include the READYMATE™ connectors (GE Healthcare) as well as the KLEENPAK™ connector (Pall Corp.).

Optionally, one or more clamping means for securing the connection is included in the autoclave bag before autoclave. Example clamping means include cable tie wraps or Snapper clamps. The connection between the tubing and the connectors is secured using a clamping means after autoclave.

Autoclave bags are readily available. One such kind of bag is the tyvex autoclave bag made from TYVEK™. These bags are lint-free, moisture resistant, puncture resistant and extremely tough. While being breathable, they provide an excellent bacterial barrier for superior performance. The bags containing the connectors and tubing can be heat sealed or taped closed. Optionally, operating instructions are printed on the outside of the bag, to direct users on how to autoclave, how to assemble the components while still inside the sterile bag, etc. When opened, the tyvex autoclave bags generate virtually no airborne particulate.

Other autoclave bags than a standard autoclave bag can also be used, as long as the following functions are fulfilled: (1) part of the bag is visible for assembly of the parts in the bag, (2) part of it contains breathable membrane, (3) it is a closed system to maintain sterility after autoclaving, and (4) flexibility in the bag wall to allow easy assembly of the parts or close the valve (see below).

In certain embodiments, the tubing and connectors are not connected before autoclaving.

In other embodiments, two connectors are included in an autoclave bag, with one tubing, at least one of the connectors is connected to the tubing before autoclave.

In one variation of the embodiment, the two connectors are each sealed by a non-breathable (non-permeable) film, and the tubing and both connectors are connected before autoclaving. It is notable that the sealed assembly of connectors and tubing does not include a vent valve, yet it could be sterilized by application of a dry autoclave cycle.

In a variation of the embodiment, at least one of the two connectors is sealed by a breathable (permeable) film, and the tubing and both connectors are connected before autoclaving.

In an alternative embodiment, two connectors are included in an autoclave bag, with one tubing, at least one of the connectors includes a vent valve which forms an integral part of the connector. Thus the tubing and connectors are connected before autoclaving with the vent valve open before autoclave. The vent valve allows steam to get into the connected assembly during autoclave. The vent valve is then closed before the sterile connected device (assembly) is taken out of the autoclave bag. The inclusion of a vent valve allows for repeated autoclavation, if needed after the first autoclavation, without the need to disassemble the system.

The methods are based on exposure of the material to be autoclaved to pressurized steam at a temperature of between about 121° C. and about 135° C., more particularly between about 121° C. and about 126° C. In a particularly preferred embodiment, the autoclave bag with the connectors and tubings are exposed to pressurized steam at a temperature of 125° C.

Generally, moist heat sterilization by autoclaving refers to heating a material in an autoclave (e.g. gravity displacement apparatus) under a pressure of at least 2 bars to achieve a temperature of between about 121° C. and about 135° C. In the sterilization process, microorganisms are killed by heating in the presence of moisture and elevated pressure. See for example, “Understanding the Operation & Validation of Autoclaves: A Practical Approach”, Reeks, B., BDR Publishing (September 1999). The sterilization period required is dependent on both the temperature and the size of the sample to be sterilized and can be in the range from 10 to 60 minutes. As the temperature and pressure are increased, the time required to achieve complete sterilization can normally be reduced, as shown in Table 1.

TABLE 1

| Temperature (° C.) | Time (minutes) | Pressure (bars, abs) |
|---|---|---|
| 121-124 | 15 | 2.01 |
| 126-129 | 10 | 2.4 |
| 134-137 | 3 | 3.05 |

In another aspect, the invention provides a kit containing the autoclave bag, tubing and connectors. Optionally, the kit contains clamping means for securing the connectors and tubing.

In one embodiment, the kit includes a tubing segment, two connectors, all sealed inside an autoclave bag. For example, the kit includes a GORE™ STA-PURE® tubing segment and two READYMATE™ aseptic connectors, sealed inside a TYVEK™ autoclave bag. Any component that is not gamma irradiation compatible can be assembled sterile post-autoclaving. Steam from autoclave will penetrate and heat-sanitize all contact surfaces. Sterility is realized irrespective of whether the tubing and connector assembly is completely sealed before autoclave. Optionally, clamping means such as cable tie wraps or Snapper clamps is included in the autoclave bag. When the bag is opened to room air the tubing connections previously made may now be secured properly by cable tie wraps or Snapper clamps.

In one embodiment, the TYVEK™ bag contains instructions, printed on the outside, for how to autoclave and assemble the tubing and connectors to ensure sterility of the completed assembly. A typical instruction would include a warning so the user does not open the bag prior to autoclave and assembly of connectors and tubing. The instruction would also include recommended autoclave settings, cooling time post autoclave, instruction on how to assemble the assembly together (for example insert the connector's tubing barb into each end of the tubing), instructions on how to open the bag and secure the tubing to the connector (using cable tie wraps or Snapper clamps).

Other features and advantages of the invention will be apparent from the following examples and from the claims.

### EXAMPLES

The present examples are provided for illustrative purposes only, and should not be construed as limiting the invention as defined by the appended claims. All references given below and elsewhere in the present specification are hereby included herein via reference.

### Example 1

GORE™ STA-PURE® tubing is not Gamma compatible, yet it is desirable for use with gamma irradiated connectors to form sterile connection assemblies. A GORE™ STA-PURE® tubing ½-in ID (24 inches in length) was connected on one side to a READYMATE™ Aseptic Connector (GE Healthcare). This was inserted, with another READYMATE™ Aseptic Connector (not connected to the GORE™ STA-PURE® tubing), into an autoclave bag (Cardinal Health Self Seal Pouch 12″×15″ Catalog 92152). The parts within the bag are visible as one side of the bag is clear tinted plastic (while paper on the other side). The bag is then heat sealed and subjected to a dry cycle at 125° C. for 15 minutes.

After autoclave, the bag is allowed to cool to room temperature. Then the READYMATE™ Aseptic Connector which was not connected before autoclave was connected with the GORE™ STA-PURE® pump tubing without the need to open the bag. The connection was easily achieved within one minute without opening the autoclave bag.

### Example 2

This experiment tested whether the READYMATE™ connectors based on non-permeable protection film becomes sterile during an autoclave procedure.

In the study, READYMATE™ connector and hose (˜1 m in length) assemblies were challenged with Geobacilllus stearothermophilus spore strips (SGM strip Geobacillus stearothermophilus 7953, 1.3×10⁶ spores/unit, SGM Biotech Inc.). One assembly was with an open end and one with a closed end by a TC stop plug. Spore strips were placed inside the double folded film on the READYMATE™ connector, under the film and 25, 50 and 75 cm from the connector in hose.

Two kinds of autoclave process was evaluated; one normally used for utensils and one preferably used for liquids. Both used the temperature of 121° C. Run time was 15 minutes. After the autoclave process the spore strips were tested for growth.

This study showed that the READYMATE™ connectors become sterile during a normal autoclave process for utensils, 121° C. 15 min. The process worked well with ˜1 m length hose and it was not necessary to use a ventilation filter at hose end.

The liquid cycle, although sterilized the inside of the READYMATE™ connector, did not kill all spores in the hoses.

All patents, patent publications, and other published references mentioned herein are hereby incorporated by reference in their entireties as if each had been individually and specifically incorporated by reference herein. While preferred illustrative embodiments of the present invention are described, one skilled in the art will appreciate that the present invention can be practiced by other than the described embodiments, which are presented for purposes of illustration only and not by way of limitation. The present invention is limited only by the claims that follow.

1. A method for providing a sterile connected device including a tubing and at least one connector, which method comprises:
(1) providing said connector and tubing in an autoclave bag, wherein said tubing is not compatible with sterilization by gamma irradiation; and
(2) autoclaving the sealed autoclave bag which allows steam penetration to reach the interior surfaces of the connector and tubing.

2. The method of claim 1, wherein said tubing is pump tubing suitable for high pressure or long term use which has a PTFE internal reinforcing layer.

3. The method of claim 2, wherein said pump tubing is a GORE™ STA-PURE® tubing.

4. The method of claim 1, wherein said at least one connector are gendered or genderless aseptic connectors.

5. The method of claim 1, wherein said at least one connector are two connectors at least one of which includes a vent valve, the tubing and connectors are connected before autoclaving, further wherein the vent valve is open before autoclave, and is closed before the sterile connected device is taken out of the autoclave bag.

6. The method of claim 1, wherein said autoclaving step is performed at a temperature of between about 121° C. and about 135° C.

7. The method of claim 1, wherein said autoclaving step is performed at a temperature of between about 121° C. and about 130° C.

8. The method of claim 1, wherein said autoclaving step is performed at a pressure designed to achieve the desired temperature.

9. The method of claim 1, wherein said autoclaving step is performed for 10 to 60 minutes.

10. The method of claim 1, wherein the tubing and connector are not connected before autoclaving.

11. The method of claim 1, wherein said at least one connector are two connectors, one of which is connected to said tubing before autoclave.

12. The method of claim 10 or 11, which method further comprises connecting said tubing with said connector before opening the bag.

13. The method of claim 12, wherein one or more clamping means for securing the connection is included in the autoclave bag before autoclave, and the connection between the tubing and the connector is secured using said clamping means after autoclave.

14. The method of claim 12, wherein a sealed unit is created after the connecting step such that interior surfaces of the unit remain sterile upon opening of the autoclave bag.

15. The method of claim 1, wherein said at least one connector are two connectors, and the tubing and connectors are connected before autoclaving.

16. The method of claim 15, wherein at least one of said connectors is breathable.

17. The method of claim 15, wherein both connectors are non-breathable.
